(19) United States
(12) Patent Application Publication
(10) Pub. No.: US 2016/0128926 A1
(43) Pub. Date: May 12, 2016

(54) COMPOSITIONS AND METHODS FOR TREATING NAILS, CLAWS, AND HOOFS

(71) Applicant: TOPICAL SOLUTIONS, INC., Carrboro, NC (US)

(72) Inventors: Mitchell DELONG, Chapel Hill, NC (US); Elise OLSEN, Chapel Hill, NC (US)

(73) Assignee: TOPICAL SOLUTIONS, INC., Carrboro, NC (US)

(21) Appl. No.: 14/890,284

(22) PCT Filed: May 8, 2014
(86) PCT No.: PCT/US2014/037312

§ 371 Date: Nov. 10, 2015

Related U.S. Application Data
(60) Provisional application No. 61/821,837, filed on May 10, 2013.

(51) Int. Cl. A61K 8/69; A61Q 3/00; A61K 8/36; A61K 8/46; A61K 8/42

(52) U.S. Cl. CPC A61K 8/69; A61K 8/46; A61K 8/42; A61K 8/36; A61Q 3/00

(57) ABSTRACT

The present approach relates to compositions and methods for treating fingernails, toenails, hoofs, claws and the like, in mammals. More particularly, the present approach relates to compositions and methods including a prostaglandin analog active ingredient and a carrier. The compositions and methods may be used for increasing the thickness, strength, integrity, length and/or growth rate of human nails, or both, and promoting healthy nail growth and appearance, as well as relates to compositions and methods for increasing the thickness or strength of horny parts of mammals, and promoting healthy nail, claw and hoof growth.

### CROSS REFERENCE TO RELATED APPLICATIONS

This application claims the benefit of U.S. Provisional Application No. 61/821,837, filed May 10, 2013, the contents of which are incorporate by reference in their entirety.

### FIELD OF THE INVENTION

The present invention relates to compositions and methods for treating fingernails, toenails, hoofs, claws and the like, in mammals. More particularly, the present invention relates to compositions and methods for increasing the thickness, strength, integrity, length and/or growth rate of human nails, or both, and promoting healthy nail growth and appearance, as well as relates to compositions and methods for increasing the thickness or strength of horny parts of mammals, including but not limited to, the nails and claws of mammals and the hoofs of ungulates, and promoting healthy nail, claw and hoof growth.

### BACKGROUND

Weak or thin fingernails, toenails, hoofs and claws, together ‘nails’, is a common problem which is, for example, naturally occurring or chemically promoted through the use of certain therapeutic drugs designed to alleviate conditions such as cancer. This leads to cracking, tearing, splitting, onychoschizia, onychorrhexis, onychomalacia, onychoclasis, peeling and the like, causing in some cases severe pain and dysfunction. Often cancer treatments are accompanied by lack of proper nail re-growth which results in nails that splinter, chip or tear easily after the cessation of such treatments as well. Excessive exposure to water, such as frequent hand washing or occupations where hands are submerged in water can cause brittle nails. Excessive wear in situations where abrasion is common is a problem as well, particularly with horses. With age, nails tend to become brittle as well, particularly in post-menopausal women. Certain disease conditions, including alopecia areata, lichen planus and twenty nail dystrophy, result in nails that are thinner than normal and in other conditions, such as diabetes, nails may have less than adequate growth rates, while other conditions, such as Nails in those with psoriasis, may have faster than normal growth rates, but poorly formed nails that are often pitted and may benefit from thickening and strengthening.

Nails may occasionally become infected by fungus which can cause them to become thickened, brittle, and to have poor separation of the nail plate and nail bed (onycholysis). Treatment for a nail fungal infection typically lasts until the entire nail grows out normally which can be a prolonged time period for toenails, especially in the elderly. A treatment that increases the nail growth rate benefits the treatment of nail fungus, as it reduces the time to normalization and lessens the need for fungal treatments, which are often poorly tolerated.

Nails may also be removed surgically because of trauma, infection or to correct defects, such as ingrown nails. Nails may also be lost because of X-ray therapy applied to the skin surface, for example, during electron beam radiation for cutaneous lymphoma. A treatment that enhances and speeds the rate of regrowth after these events is an important advancement.

Fingernails and toenails are composed of protein fibers called keratin which is the same protein hair is made of. Nail growth occurs from the posterior nail fold forward to the distal border of the lunula, the moon shaped area seen at the base of many nails: collectively this is sometimes called the nail growth plate. As the nail grows, cells from the matrix multiply, mature, and die. Upon death, they become filled with this protein matrix which gives the nail its tough, hard exterior. Many factors can affect the rate of nail growth, including the blood supply and the age of the patient.

While nails that are weak, brittle, or otherwise unsuitable is a condition that affects millions, there are few treatments available to remedy this condition. Those suffering from this condition, other than those with a nail fungal infection, are offered very little. “Eat a good diet; get plenty of sleep” and other such generic advice is frequently the only proffered solution. Nail salons offer only covering the nails with chemicals that bond to the nail and act as a physical scaffold, such as a nail polish or a nail gel, many of which are polymeric, such as polyacrylates. They also glue false nails on top of the subjects' nail plates to enhance the appearance. These treatments do nothing to address the cause of the weak nails and in fact may further weaken the nails, especially with the necessary reapplication at regular intervals, causing women who use these methods to be “a slave to their nail salon”. These methods can cost more than $1,000 dollars per year just to maintain the look of healthy nails, while in reality, diminishing the health of the nails. Nails have several functions that can be severely impaired by nail diseases. Fingernails are necessary for manipulating objects and for tactile sensation, but they are also an important communication tool. Patients with severe fingernail abnormalities may have great problems in their social life and often try to hide their hands from sight. In addition, strong healthy nails can aid in defense in some situations.

However, despite the need, there is a lack of products available to treat weak nails. Instead of pharmaceutical agents, people are reduced to trying home remedies of uncertain effect. For example, suggested treatments for weak nails include taking Gelatin and/or biotin capsules, massaging almond, olive oil, or cocoa butter into the cuticles, taking the dietary supplement MSM (methylsulfonylmethane), painting nails nightly with “de-colorized iodine”, using superglue on cracked nails, applying petroleum jelly on nails, massaging glycerin into nails, taking 5000 units a day of Vitamin D₃ cholecalciferol, taking a digestive enzyme with Betaine HCL to help assimilate mineral supplements, drinking 1 tablespoon of apple cider vinegar mixed with a glass of tomato or other vegetable juice, a diet including raw spinach and cooked yams, taking one tablespoon each of apple cider, vinegar, and honey daily, drinking a cup of blackstrap molasses each morning with the juice of half a lemon, taking fish oil supplements, taking a folic acid supplement, dipping fingernails in colorless iodine, applying juice from a lemon to the fingernails every day, and “taking silica supplements” (which means essentially eating sand).

In light of the problems associated with weak or thin nails, there is an ongoing and present need for effective, new compounds and methods of treating nails. It has been surprisingly discovered that certain prostaglandins are effective at promoting nail growth, and increasing the strength and thickness and fitness of nails. Weak, thin, slowly-growing, brittle, pitted nails are surprisingly made thicker and stronger upon sustained treatment. These prostaglandins are thus useful for the treatment of cracking, tearing, splitting, onychoschizia, onychorrhexis, onychomalacia, peeling and the like.

Naturally occurring prostaglandins (e.g., PGA₂, PGB₂, PGE₁, PGE₂, PGF_{2α}, and PGI₂) are C-20 unsaturated fatty acids. PGF_{2α}, the naturally occurring Prostaglandin F analog in humans, is characterized by hydroxyl groups at the C₉ and C₁₁ positions on the alicyclic ring, a cis-double bond between C₅ and C₆, and a trans-double bond between C₁₃ and C₁₄. PGF_{2α}, has the formula:

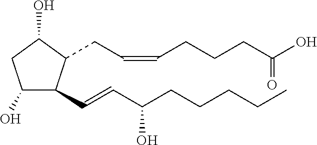 [SMILES: CCCCC[C@H](O)/C=C/[C@@H]1[C@@H](C/C=C\CCCC(=O)O)[C@@H](O)C[C@H]1O]

Wherein the “2” in the name indicates the number of alkenes in the structure. PGF₀, PGF₁, PGF₂ and PGF₃ compounds are all known in the art. When the alkenes are in the 5,6 and/or the 13,14 position, no specific reference is made to their location. When they are in other locations, note that 5,6-dihydro PGF₂ is the same structure as is PGF₁. The “alpha” (α) symbol indicates that the hydroxyl moiety at C-9 has an alpha (behind the plane of the paper) configuration when displayed in the ‘standard” display (as shown above). Many suitable analogs of the prostaglandins of the F series are known in the art.

Nomenclature of the prostaglandins varies considerably from reference to reference, with trivial, prostanoid-specific standard, and IUPAC standard and historical names all being used in the art. All of these are used interchangeably herein.

Prostaglandins in general have a wide range of biological activities. For example, they have the following properties: a) regulator of cell proliferation; b) regulator of cytokine synthesis; c) regulator of immune responses; and d) inducer of vasodilatation. A lack of sufficient blood supply being one of the theories to explain why nails grow more slowly in winter than in summer. In vitro results in the literature also indicate some anti-inflammatory properties of the prostaglandins.

However, no attempts at using prostaglandins to promote nail growth have been reported in the literature, despite their use for many other conditions. Many experts hold that topical treatment, of any kind, is impractical as the nail bed is too deeply seated to have sufficient access from the topical application. “Topical treatments are not as successful on the nails as on the skin because the nail plate prevents drug penetration, and the nail matrix is deeply located under the proximal nail fold” (TREATMENT OF COMMON NAIL DISORDERS, Dermatologic Clinics, Volume 18, Issue 2, Pages 339-348 Antonella Tosti, Bianca Maria Piraccini).

A further complication is that different prostaglandin analogs can bind to multiple receptors at various concentrations with a biphasic effect. Also, the availability of in vitro animal models to predict human efficacy is largely lacking. This makes evaluating the animal data to predict human efficacy challenging.

Therefore, it is an object of the present invention to provide methods for using prostaglandin analogs to grow nails, hoofs and claws and to provide compositions that promote nail and hoof growth. It is a further object of this invention to provide a selection of appropriate prostaglandin analogs that will promote nail and hoof growth.

### SUMMARY OF THE INVENTION

Disclosed is a composition and method for treating nail weakness. A method, for example, can provide for administering a composition comprising a specific prostaglandin analog that interacts strongly with receptors found in the nail bed, such as the FP receptor. The choice of prostaglandin analog is critical because the prostaglandin analog must selectively activate the nail growth-promoting receptor(s) without activating any other receptors that would negate the effect of activating the nail growth-promoting receptor(s).

In one embodiment, a topical composition for treating nail weakness and increasing nail thickness, integrity, strength, growth rate, and/or appearance comprises: (a) a prostaglandin analog, wherein the prostaglandin analog is the active ingredient; and (b) a carrier. The topical composition may further include an optional activity enhancer. Suitable prostaglandin analogs for the present invention have the general formula:

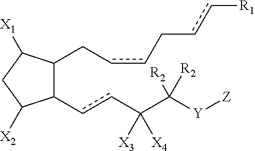

The dashed lines in the structure indicate optionally single, double, or triple bonds. If the bonds are double bonds, they may be in the cis or in the trans configuration. Other suitable prostaglandins are pharmaceutically acceptable salts, epimers, hydrates, and biohydrolyzable amides, esters, and imides of the general formula above. Optical isomers, diastereomers, and enantiomers of the structure described below are also suitable for this invention. At all stereocenters where stereochemistry is not defined, both epimers are envisioned with the epimer that corresponds to the naturally-occurring one being preferred.

In another embodiment of the topical composition, R¹ in the prostaglandin analog can be selected from the group consisting of CO₂H, C(O)NHR⁵, CO₂R⁵, CH₂OH, S(O)₂R⁵, P(O)MeOR⁵, C(O)NHR⁵, C(O)NHS(O)₂R⁵, and tetrazole. R⁵ can be selected from the group consisting of monovalent hydrocarbon groups, substituted monovalent hydrocarbon groups, aromatic groups, substituted aromatic groups, carbocyclic groups, substituted carbocyclic groups, heterogeneous groups, substituted heterogeneous groups, heterocyclic groups, substituted heterocyclic groups, heteroaromatic groups, and substituted heteroaromatic groups. In a preferred embodiment of the topical composition of the present invention, R⁵ can be selected from the group consisting of CH₃, C₂H₅, and C₃H₇. In another embodiment of the topical composition of the present invention, R¹ can be selected from the group consisting of CO₂H, CO₂CH₃, CO₂C₂H₅, CO₂C₃H₇, CO₂C₄H₉, CO₂C₃H₇O₂, and C(O)NHR⁵. In another embodiment, R¹ can be selected from the group consisting of CO₂H, CO₂CH₃, CO₂C₂H₅, C(O)NHR⁵ and CO₂C₃H₇. In another embodiment, R¹ can be selected from the group consisting of CO₂H, CO₂CH₃, C(O)NHR⁵ and CO₂C₃H₇. In an embodiment of the composition of the present invention, R¹ is C(O)NHR⁵ and R⁵ can be selected from the group consisting of CH₃ and C₂H₅.

In an embodiment of the composition of the present invention, each R² in the prostaglandin analog compound can independently be a hydrogen atom or a lower monovalent hydrocarbon group.

In an embodiment of the composition, X₁, X₂, X₃ and X₄ in the prostaglandin analog compound are each independently selected from the group consisting of H, halogen, CH₃, C₂H₅, NR²R², OR¹⁰, SR¹⁰, and OH; with the proviso that both X₃ and X₄ are not OH. In other words, in some embodiments, when X₃ is OH, then X₄ is selected from the group consisting of H, halogen, CH₃, C₂H₅, NR²R², OR¹⁰, and SR¹⁰. R¹⁰ is selected from the group consisting of a monovalent hydrocarbon group, a substituted monovalent hydrocarbon group, a heterogeneous group, a substituted heterogeneous group, a carbocyclic group, a substituted carbocyclic group, an aromatic group, a substituted aromatic group, a heteroaromatic group, and a substituted heteroaromatic group; with the proviso that R¹⁰ has 1 to 8 member atoms. In an embodiment of the composition of the present invention, X₁, X₂, and X₃ are OH and X₄ is a hydrogen atom, or X₃ and X₄ are both F.

In an embodiment of the composition of the present invention, Y can be selected from the group consisting of an oxygen atom, a divalent hydrocarbon group, a sulfur-containing moiety, and a nitrogen-containing group. The divalent hydrocarbon group has the formula (CH₂)ₙ, wherein n is an integer with a value of 0 to 5. Preferably, n is 0, 1, or 2; more preferably, n is 1.

In an embodiment wherein Y is a sulfur-containing moiety, the sulfur-containing moiety for Y can be selected from the group consisting of a sulfur atom, S(O), and S(O)₂. If Y is a sulfur-containing moiety, it can be a sulfur atom.

In an embodiment wherein Y is a nitrogen-containing group, the nitrogen-containing group for Y can have the formula NR¹¹. R¹¹ can be selected from the group consisting of a hydrogen atom, an acyl group, a monovalent hydrocarbon group, a substituted monovalent hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group. Preferably R¹¹ is H or CH₃.

An aspect of some embodiments of the composition is that Z can be selected from the group consisting of a carbocyclic group, a substituted carbocyclic group, a heterocyclic group, a substituted heterocyclic group, an aromatic group, a substituted aromatic group, a heteroaromatic group, and a substituted heteroaromatic group. In an embodiment, Z can be selected from the group consisting of a monocyclic carbocyclic group, a substituted monocyclic carbocyclic group, a monocyclic heterocyclic group, a substituted monocyclic heterocyclic group, a monocyclic aromatic group, a substituted monocyclic aromatic group, a monocyclic heteroaromatic group, and a substituted monocyclic heteroaromatic group. In another embodiment, Z can be selected from the group consisting of a monocyclic aromatic group, a substituted monocyclic aromatic group, a monocyclic heteroaromatic group, and a substituted monocyclic heteroaromatic group. In one embodiment, Z is thienyl or phenyl.

### DETAILED DESCRIPTION

Compositions comprising prostaglandin F analogs (“prostaglandins”) can be used to treat nail weakness or to enhance nail thickness, integrity, strength and/or growth rate in mammals. “Treating nail weakness” includes arresting nail weakness or reversing nail weakness, or both, and promoting nail growth.

Publications and patents are referred to throughout this disclosure. All U.S. patents cited herein are hereby incorporated by reference.

All percentages, ratios, and proportions used herein are by weight unless otherwise specified.

In the description, various embodiments and individual features are disclosed. As will be apparent to a person having ordinary skill in the art, all combinations of such embodiments and features are possible and can result in preferred embodiments of the invention.

It is understood that the present invention is not limited to the particular compositions described herein, as these may vary. Thus, for example, a reference to an ingredient in the form of a particular compound includes equivalents thereof known to those skilled in the art.

### DEFINITION AND USAGE OF TERMS

The following is a list of definitions for terms, as used herein:

“Nail” or “Nails”, as used herein means any horny tissue of a mammal, including but not limited to, horns, hoofs, claws, fingernails, and toenails.

“Aromatic group” means a monovalent group having a monocyclic ring structure or fused bicyclic ring structure. Monocyclic aromatic groups contain 5 to 10 carbon atoms, preferably 5 to 7 carbon atoms, and more preferably 5 to 6 carbon atoms in the ring. Bicyclic aromatic groups contain 8 to 12 carbon atoms, preferably 9 or 10 carbon atoms in the ring. Aromatic groups are unsubstituted. The most preferred aromatic group is phenyl.

“Carbocyclic group” means a monovalent saturated or unsaturated hydrocarbon ring. Carbocyclic groups are monocyclic, or are fused, spiro, or bridged bicyclic ring systems. Monocyclic carbocyclic groups contain 4 to 10 carbon atoms, preferably 4 to 7 carbon atoms, and more preferably 5 to 6 carbon atoms in the ring. Bicyclic carbocyclic groups contain 8 to 12 carbon atoms, preferably 9 to 10 carbon atoms in the ring. Carbocyclic groups are unsubstituted. Preferred carbocyclic groups include cyclopentyl, cyclohexyl, cyclohexenyl, cycloheptyl, and cyclooctyl. More preferred carbocyclic groups include cyclohexyl, cycloheptyl, and cyclooctyl. The most preferred carbocyclic group is cycloheptyl. Carbocyclic groups are not aromatic.

“Halogen atom” means F, Cl, Br, or I. Preferably, the halogen atom is F, Cl, or Br; more preferably Cl or F; and most preferably F.

“Halogenated hydrocarbon group” means a substituted monovalent hydrocarbon group or a substituted carbocyclic group, wherein at least one substituent is a halogen atom. Halogenated hydrocarbon groups can have a straight, branched, or cyclic structure. Preferred halogenated hydrocarbon groups have 1 to 12 carbon atoms, more preferably 1 to 6 carbon atoms, and most preferably 1 to 3 carbon atoms. Preferred halogen atom substituents are Cl and F. The most preferred halogenated hydrocarbon group is trifluoromethyl.

“Heteroaromatic group” means an aromatic ring containing carbon and 1 to 4 heteroatoms in the ring. Heteroaromatic groups are monocyclic or fused bicyclic rings. Monocyclic heteroaromatic groups contain 5 to 10 member atoms (i.e., carbon and heteroatoms), preferably 5 to 7, and more preferably 5 to 6 in the ring. Bicyclic heteroaromatic rings contain 8 to 12 member atoms, preferably 9 or 10 in the ring. Heteroaromatic groups are unsubstituted. Preferred heteroaromatic groups include thienyl, thiazolo, purinyl, pyrimidyl, pyridyl, and furanyl. More preferred heteroaromatic groups include thienyl, furanyl, and pyridyl. The most preferred heteroaromatic group is thienyl.

“Heteroatom” means an atom other than carbon in the ring of a heterocyclic group or the chain of a heterogeneous group. Preferably, heteroatoms are selected from the group consisting of nitrogen, sulfur, and oxygen atoms. Groups containing more than one heteroatom may contain different heteroatoms.

“Heterocyclic group” means a saturated or unsaturated ring structure containing carbon and 1 to 4 heteroatoms in the ring. No two heteroatoms are adjacent in the ring. Heterocyclic groups are not aromatic. Heterocyclic groups are monocyclic, or are fused or bridged bicyclic ring systems. Monocyclic heterocyclic groups contain 4 to 10 member atoms (i.e., including both carbon atoms and at least 1 heteroatom), preferably 4 to 7, and more preferably 5 to 6 in the ring. Bicyclic heterocyclic groups contain 8 to 12 member atoms, preferably 9 or 10 in the ring. Heterocyclic groups are unsubstituted. Preferred heterocyclic groups include piperzyl, morpholinyl, tetrahydrofuranyl, tetrahydropyranyl, and piperdyl.

“Heterogeneous group” means a saturated or unsaturated chain containing 1 to 18 member atoms (i.e., including both carbon and at least one heteroatom). No two heteroatoms are adjacent. Preferably, the chain contains 1 to 12 member atoms, more preferably 1 to 6, and most preferably 1 to 4. The chain may be straight or branched. Preferred branched heterogeneous groups have one or two branches, preferably one branch. Preferred heterogeneous groups are saturated. Unsaturated heterogeneous groups have one or more double bonds, one or more triple bonds, or both. Preferred unsaturated heterogeneous groups have one or two double bonds or one triple bond. More preferably, the unsaturated heterogeneous group has one double bond. Heterogeneous groups are unsubstituted.

“Lower monovalent hydrocarbon group” means a monovalent hydrocarbon group having 1 to 6, preferably 1 to 4, carbon atoms.

“Monovalent hydrocarbon group” means a chain of 1 to 18 carbon atoms, preferably 1 to 12, more preferably 1 to 6, and most preferably 1 to 4 carbon atoms. Monovalent hydrocarbon groups may have a straight chain or branched chain structure. Preferred monovalent hydrocarbon groups have one or two branches, preferably 1 branch. Preferred monovalent hydrocarbon groups are saturated. Unsaturated monovalent hydrocarbon groups have one or more double bonds, one or more triple bonds, or combinations thereof. Preferred unsaturated monovalent hydrocarbon groups have one or two double bonds or one triple bond; more preferred unsaturated monovalent hydrocarbon groups have one double bond.

“Pharmaceutically acceptable” means suitable for use in a human or other mammal.

“Substituted aromatic group” means an aromatic group wherein 1 to 4 of the hydrogen atoms bonded to carbon atoms in the ring have been replaced with other substituents. Preferred substituents include: halogen atoms, cyano groups, monovalent hydrocarbon groups, substituted monovalent hydrocarbon groups, heterogeneous groups, aromatic groups, substituted aromatic groups, or any combination thereof. More preferred substituents include halogen atoms, monovalent hydrocarbon groups, and substituted monovalent hydrocarbon groups. Preferred substituted aromatic groups include naphthyl. The substituents may be substituted at the ortho, meta, or para position on the ring, or any combination thereof.

“Substituted carbocyclic group” means a carbocyclic group wherein 1 to 4 hydrogen atoms bonded to carbon atoms in the ring have been replaced with other substituents. Preferred substituents include: halogen atoms, cyano groups, monovalent hydrocarbon groups, monovalent heterogeneous groups, substituted monovalent hydrocarbon groups, aromatic groups, substituted aromatic groups, or any combination thereof. More preferred substituents include halogen atoms and substituted monovalent hydrocarbon groups. Carbocyclic group does not include aromatic rings.

“Substituted heteroaromatic group” means a heteroaromatic group wherein 1 to 4 hydrogen atoms bonded to carbon atoms in the ring have been replaced with other substituents. Preferred substituents include: halogen atoms, cyano groups, monovalent hydrocarbon groups, substituted monovalent hydrocarbon groups, heterogeneous groups, substituted heterogeneous groups, phenyl groups, phenoxy groups, or any combination thereof. More preferred substituents include halogen atoms, halogenated hydrocarbon groups, monovalent hydrocarbon groups, and phenyl groups.

“Substituted heterocyclic group” means a heterocyclic group wherein 1 to 4 hydrogen atoms bonded to carbon atoms in the ring have been replaced with other substituents. Preferred substituents include: halogen atoms, cyano groups, monovalent hydrocarbon groups, substituted monovalent hydrocarbon groups, heterogeneous groups, substituted heterogeneous groups, halogenated hydrocarbon groups, phenyl groups, phenoxy groups, or any combination thereof. More preferred substituents include halogen atoms and halogenated hydrocarbon groups. Substituted heterocyclic groups are not aromatic.

“Substituted heterogeneous group” means a heterogeneous group, wherein 1 to 4 of the hydrogen atoms bonded to carbon atoms in the chain have been replaced with other substituents. Preferred substituents include halogen atoms, hydroxy groups, alkoxy groups (e.g., methoxy, ethoxy, propoxy, butoxy, and pentoxy), aryloxy groups (e.g., phenoxy, chlorophenoxy, tolyloxy, methoxyphenoxy, benzyloxy, alkyloxycarbonylphenoxy, and acyloxyphenoxy), acyloxy groups (e.g., propionyloxy, benzoyloxy, and acetoxy), carbamoyloxy groups, carboxy groups, mercapto groups, alkylthio groups, acylthio groups, arylthio groups (e.g., phenylthio, chlorophenylthio, alkylphenylthio, alkoxyphenylthio, benzylthio, and alkyloxycarbonylphenylthio), aromatic groups (e.g., phenyl and tolyl), substituted aromatic groups (e.g., alkoxphenyl, alkoxycarbonylphenyl, and halophenyl), heterocyclic groups, heteroaromatic groups, and amino groups (e.g., amino, mono- and di-alkylamino having 1 to 3 carbon atoms, methylphenylamino, methylbenzylamino, alkanylamido groups of 1 to 3 carbon atoms, carbamamido, ureido, and guanidino).

“Substituted monovalent hydrocarbon group” means a monovalent hydrocarbon group wherein 1 to 4 of the hydrogen atoms bonded to carbon atoms in the chain have been replaced with other substituents. Preferred substituents include halogen atoms; halogenated hydrocarbon groups; alkyl groups (e.g., methyl, ethyl, propyl, and butyl); hydroxy groups; alkoxy groups (e.g., methoxy, ethoxy, propoxy, butoxy, and pentoxy); aryloxy groups (e.g., phenoxy, chlorophenoxy, tolyloxy, methoxyphenoxy, benzyloxy, alkyloxycarbonylphenoxy, and acyloxyphenoxy); acyloxy groups (e.g., propionyloxy, benzoyloxy, and acetoxy); carbamoyloxy groups; carboxy groups; mercapto groups; alkylthio groups; acylthio groups; arylthio groups (e.g., phenylthio, chlorophenylthio, alkylphenylthio, alkoxyphenylthio, benzylthio, and alkyloxycarbonylphenylthio); aryl groups (e.g., phenyl, tolyl, alkoxyphenyl, alkoxycarbonylphenyl, and halophenyl); heterocyclyl groups; heteroaryl groups; and amino groups (e.g., amino, mono- and di-alkanylamino groups of 1 to 3 carbon atoms, methylphenylamino, methylbenzylamino, alkanylamido groups of 1 to 3 carbon atoms, carbamamido, ureido, and guanidino).

### Compositions

Disclosed is a composition for treating nail weakness or for enhancing nail thickness, integrity, strength, growth rate and/or appearance. “Treating nail weakness” means arresting nail weakness, reversing nail weakness, or both, and/or promoting nail growth. An embodiment of the composition of the present invention comprises: A) a prostaglandin; and B) a carrier. Pharmaceutically acceptable salts, hydrates, and biohydrolyzable amides, esters, and imides thereof are also suitable for component A). The composition can further comprise C) one or more optional activity enhancers.

In one embodiment of the composition, component A) can be a prostaglandin having the structure:

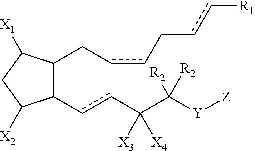

In an embodiment of the prostaglandin analog having the above structure, R¹ can be selected from the group consisting of CO₂H, C(O)NHR⁵, CO₂R⁵, CH₂OH, S(O)₂R⁵, P(O)MeOR⁵, C(O)NHR⁵, C(O)NHS(O)₂R⁵, and tetrazole. Preferably, R¹ is selected from the group consisting of CO₂H, CO₂CH₃, CO₂C₂H₅, CO₂C₃H₇, CO₂C₄H₉, CO₂C₃H₇O₂, and C(O)NHR⁵. R¹ can also be selected from the group consisting of CO₂H, CO₂CH₃, CO₂C₂H₅, C(O)NHR⁵ and CO₂C₃H₇. Most preferably, R¹ is selected from the group consisting of CO₂H, CO₂CH₃, C(O)NHR⁵ and CO₂C₃H₇.

In an embodiment of the composition, R⁵ can be selected from the group consisting of monovalent hydrocarbon groups, substituted monovalent hydrocarbon groups, aromatic groups, substituted aromatic groups, carbocyclic groups, substituted carbocyclic groups, heterogeneous groups, substituted heterogeneous groups, heterocyclic groups, substituted heterocyclic groups, heteroaromatic groups, and substituted heteroaromatic groups. R⁵ can also be selected from the group consisting of CH₃, C₂H₅, and C₃H₇. In an embodiment of the present invention, R⁵ can be selected from the group consisting of H, CH₃, C₂H₅, and C₃H₇. In an embodiment of the composition of the present invention, R¹ is C(O)NHR⁵ and R⁵ is selected from the group consisting of CH₃, C₂H₅, and C₃H₇.

In an embodiment of the composition of the present invention, each R² in the prostaglandin analog can independently be a hydrogen atom or a lower monovalent hydrocarbon group. In another embodiment, each R² in the prostaglandin analog can be selected from the group consisting of a hydrogen atom and a methyl group.

In an embodiment of the composition, X₁, X₂, X₃ and X₄ are each independently selected from the group consisting of H, halogen, CH₃, C₂H₅, NR³R⁴, OR¹⁰, SR¹⁰, and OH; with the proviso that both X₃ and X₄ are not OH, but X₃ and X₄ may combine to be a ketone moiety or a ketal. R³, R⁴ and R¹⁰ can be independently selected from the group consisting of a monovalent hydrocarbon group, a substituted monovalent hydrocarbon group, a heterogeneous group, a substituted heterogeneous group, a carbocyclic group, a substituted carbocyclic group, an aromatic group, a substituted aromatic group, a heteroaromatic group, and a substituted heteroaromatic group, or hydrogen. In an embodiment of the composition, R³ and R⁴ are both hydrogen atoms. In an embodiment of the composition, R¹⁰ is selected from the group consisting of CH₃ and C₂H₅.

In another embodiment of the composition of the present invention, X₁, X₂, and X₃ are each independently selected from the group consisting of H, F, OH, CH₃, and C₂H₅. In one embodiment of the composition, X₁, X₂, and X₃ are OH and X₄ is a hydrogen atom, or X₃ and X₄ are both F. In an embodiment of the composition, X₁ and X₂ are both OH moieties. In another embodiment, X₃ is selected from the group consisting of NR³R⁴, and OR¹⁰. In an embodiment of the composition, Y can be selected from the group consisting of an oxygen atom, a divalent hydrocarbon group, a sulfur-containing moiety, and a nitrogen-containing group. The divalent hydrocarbon group has the formula (CH₂)ₙ, wherein n is an integer with a value of 0 to 5. Preferably, n is 0, 1, or 2; more preferably, n is 1.

In an embodiment wherein Y is a sulfur-containing moiety, the sulfur-containing moiety for Y can be selected from the group consisting of a sulfur atom, S(O), and S(O)₂. When Y is a sulfur-containing moiety, it preferably is a sulfur atom.

In an embodiment wherein Y is a nitrogen-containing group, the nitrogen-containing group for Y has the formula NR¹¹. R¹¹ can be selected from the group consisting of a hydrogen atom, an acyl group, a monovalent hydrocarbon group, a substituted monovalent hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group. In another embodiment, R¹¹ is H or CH₃.

In an embodiment of the composition, Z can be selected from the group consisting of a carbocyclic group, a substituted carbocyclic group, a heterocyclic group, a substituted heterocyclic group, an aromatic group, a substituted aromatic group, a heteroaromatic group, and a substituted heteroaromatic group. Z can also be selected from the group consisting of a monocyclic carbocyclic group, a substituted monocyclic carbocyclic group, a monocyclic heterocyclic group, a substituted monocyclic heterocyclic group, a monocyclic aromatic group, a substituted monocyclic aromatic group, a monocyclic heteroaromatic group, and a substituted monocyclic heteroaromatic group. In an embodiment, Z can be selected from the group consisting of a monocyclic aromatic group, a substituted monocyclic aromatic group, a monocyclic heteroaromatic group, and a substituted monocyclic heteroaromatic group. In another embodiment, Z is thienyl or phenyl.

The dashed lines in the prostaglandin analog general structure above indicate optionally single, double, or triple bonds. If the bonds are double bonds, they may be in the cis or in the trans configuration. Optical isomers, diastereomers, and enantiomers of the structure described above are also suitable as component A) of this invention. At all stereocenters where stereochemistry is not defined, both epimers are envisioned.

Examples of suitable prostaglandins having the formula above include, but are not limited to:
16-(3-methylphenylthio)tetranor Prostaglandin F_{2α} methyl ester;
13,14-dihydro-16-(3-methylphenylthio)tetranor Prostaglandin F_{1α};
16-(3-fluorophenylthio)tetranor Prostaglandin F_{2α} methyl ester;
13,14-dihydro-16-(3-fluorophenylthio)tetranor Prostaglandin F_{1α} isopropyl ester
13,14-dihydro-16-(2,6-difluorophenylthio)tetranor Prostaglandin F_{1α} methyl ester;
16-(3,5-difluorophenylthio)tetranor Prostaglandin F_{2α};
13,14-dihydro-16-(2-methylphenylthio)tetranor Prostaglandin F_{1α} N-ethyl amide;
13,14-dihydro-16-(4-methylphenylthio)tetranor Prostaglandin F_{1α} methyl ester;
13,14-dihydro-16-(4-methylphenylthio)tetranor Prostaglandin F_{1α};
13,14-dihydro-16-(2-fluorophenylthio)tetranor Prostaglandin F_{1α} glyceryl ester;
16-(2-fluorophenylthio)tetranor Prostaglandin F_{2α};
13,14-dihydro-15-methyl-16-(3-fluorophenylthio)tetranor Prostaglandin F_{1α} methyl ester;
13,14-dihydro-16-(2-fluorophenylthio)tetranor prostaglandin F_{1α} 1-hydroxamic acid;
16-(3-chlorophenylamino)tetranor prostaglandin F_{2α} 1-hydroxamic acid;
13,14-dihydro-16-(3-trifluoromethylphenylthio)tetranor Prostaglandin F_{1α} methyl ester;
13,14-dihydro-16-(3-trifluoromethylphenylthio)tetranor Prostaglandin F_{1α};
13,14-dihydro-16-(3-trifluoromethylphenylthio)tetranor prostaglandin F_{1α} 1-hydroxamic acid;
13,14-dihydro-16-(phenylthio)tetranor Prostaglandin F_{1α} methyl ester;
15,15-difluoro-16-(phenylthio)tetranor Prostaglandin F_{2α}; isopropyl ester;
13,14-dihydro-15-methyl-16-(phenylthio)tetranor Prostaglandin F_{1α} methyl ester;
13,14-dihydro-15-methyl-16-(phenylthio)tetranor Prostaglandin F_{1α};
13,14-dihydro-16-(phenylamino)tetranor PG F_{1α}α methyl ester;
13,14-dihydro-16-(phenylamino)tetranor prostaglandin F_{1α};
13,14-dihydro-16-(2-thienylthio)tetranor prostaglandin F_{1α} ethyl ester;
13,14-dihydro-16-(2-thienylthio)tetranor prostaglandin F_{1α};
13,14-dihydro-16-(1-napthylthio)tetranor Prostaglandin F_{1α} isopropyl ester;
13,14-dihydro-16-(1-napthylthio)tetranor Prostaglandin F_{1α};
13,14-dihydro-15-butoxy-15-dehydroxy-16-(phenylthio)tetranor prostaglandin F_{1α} methyl ester;
17-phenyl-17-trinor prostaglandin F_{2α} methyl ester;
17-phenyl 17-trinor PGF_{2α} cyclopropyl methyl amide;
17-(2,4-difluorophenyl)-17-trinor prostaglandin F_{2α};
13,14-dihydro-17-(2-fluorophenyl)-17-trinor prostaglandin F_{1α} methyl ester;
5,6-dihydro-17-(2-fluorophenyl)-17-trinor prostaglandin F_{1α} isopropyl ester;
13,14-dihydro-17-(3-fluorophenyl)-17-trinor prostaglandin F_{1α} methyl ester;
17-(3-trifluoromethylphenyl)-17-trinor prostaglandin F_{2α};
17-(3-trifluoromethylphenyl)-17-trinor prostaglandin F_{2α} methyl ester;
18-(2-thienyl)-18-dinor prostaglandin F_{2α} methyl ester;
13,14-dihydro-18-(2-thienyl)-18-dinor PGF_{1α} isopropyl ester;
5,6-dihydro-17-((2-trifluoromethyl)phenyl)-17-trinor prostaglandin F_{1α} methyl ester;
13,14-dihydro-17-((2-trifluoromethyl)phenyl)-17-trinor prostaglandin F_{1α};
13,14-dihydro-17-((3-trifluoromethyl)phenyl)-17-trinor prostaglandin F_{1α} n-butyl ester;
13,14-dihydro-17-((3-trifluoromethyl)phenyl)-17-trinor prostaglandin F_{1α};
13,14-dihydro-17-((4-trifluoromethyl)phenyl)-17-trinor prostaglandin F_{1α} cyclohexyl ester;
13,14-dihydro-17-((4-trifluoromethyl)phenyl)-17-trinor prostaglandin F_{1α};
13,14-dihydro-17-(2-methylphenyl)-17-trinor prostaglandin F_{1α} N-methyl amide;
13,14-dihydro-17-(2-methylphenyl)-17-trinor prostaglandin F_{1α};
13,14-dihydro-17-(3-methylphenyl)-17-trinor prostaglandin F_{1α} N-ethyl amide;
13,14-dihydro-17-(3-methylphenyl)-17-trinor prostaglandin F_{1α};
13,14-dihydro-17-(4-methylphenyl)-17-trinor prostaglandin F_{1α} N-propyl amide ester;
13,14-dihydro-17-(4-methylphenyl)-17-trinor prostaglandin F_{1α}.

Examples of suitable prostaglandin's having the general formula above, wherein Y is an oxygen atom include, but are not limited to:
13,14-dihydro-16,16-dimethyl-16-(2-fluorophenoxy)-16-tetranor prostaglandin F_{2α};
13,14-dihydro-16,16-dimethyl, 16-(2-methylphenoxy)-16-tetranor Prostaglandin F_{1α};
13,14-dihydro-16,16-dimethyl, 16-(2,3 difluorophenoxy)-16-tetranor Prostaglandin F_{1α};
20-ethyl Prostaglandin F_{2α};
13,14-dihydro-, 16-(3-fluoro-5-trifluoromethyl phenoxy)-16-tetranor Prostaglandin F_{1α};
isopropyl ester;
13,14-dihydro-16,16-dimethyl, 16-(4-chlorophenoxy)-16-tetranor Prostaglandin F_{2α};
13,14-dihydro-16-methyl, 16-(3-chlorophenoxy)-16-tetranor Prostaglandin F_{1α} isopropyl ester;
13,14-dihydro-16-isopropyl-16-(2-fluorophenoxy)-16-tetranor prostaglandin F_{1α};
13,14-dihydro-16-ethyl, 16-(2-methylphenoxy)-16-tetranor Prostaglandin F_{1α} isopropyl ester
13,14-dihydro-16-(hydroxymethyl)-16-phenoxy-16-tetranor Prostaglandin F_{1α} N-ethyl amide;
13,14-dihydro-16-methyl-16-(4-ethylphenoxy)-16-tetranor Prostaglandin F_{1α};
16-methyl-16-(3-chlorophenoxy)-16-tetranor Prostaglandin F_{1α} methyl ester;
13,14-dihydro-16-methyl-16-(4-phenylphenoxy)-16-tetranor Prostaglandin F_{1α} isopropyl ester;
13,14-dihydro-16,16-dimethyl-16-(4-phenoxyphenoxy)-16-tetranor Prostaglandin F_{1α} isopropyl ester;
16,16-dimethyl-16-(2-fluorophenoxy)-16-tetranor Prostaglandin F_{2α} hydroxamic acid;
16-methyl-16-(3-chlorophenoxy)-tetranor Prostaglandin F_{2α} 1-hydroxamic acid;
13,14-dihydro-16-methoxymethyl-16-(2,3-difluorophenoxy)-16-tetranor Prostaglandin F_{1α} 1-hydroxamic acid;
13,14-dihydro-16-(phenoxy)tetranor Prostaglandin F_{1α}, methanesulfonamide;
13,14-dihydro-15-dehydroxy-15-fluoro-16-(2-fluorophenoxy)tetranor Prostaglandin F_{1α} methyl ester;
13,14-dihydro-15-methyl-16,16-dimethyl-16-(2-fluorophenoxy)-16-tetranor Prostaglandin F_{1α};
13,14-dihydro-15-dehydroxy-15-fluoro-16-(2,3-difluorophenoxy)-16-tetranor Prostaglandin F_{1α}1-hydroxamic acid;
13,14-dihydro-15-methylthio-15-dehydroxy-16-(2-methylphenoxy)-16-tetranor Prostaglandin F_{1α};
5,6-dihydro-15-methylthio-15-dehydroxy-16-methyl-16-(2-methylphenoxy) 16-tetranor Prostaglandin F_{1α} 1-hydroxamic acid;
15-methoxy-16,16-dimethyl-16-(2-fluorophenoxy)-16-tetranor Prostaglandin F_{2α} 1-hydroxamic acid;
13,14-dihydro-15-ethoxy-15-dehydroxy-16-phenoxy-16-tetranor Prostaglandin F_{1α} isopropyl ester;
13,14-dihydro-15-sulfonylmethyl-15-dehydroxy-16-methyl-16-(2-methylphenoxy)-tetranor Prostaglandin F_{1α} methyl ester; 15-sulfoxylmethyl-15-dehydroxy-16-methyl-16-(2-methylphenoxy)-tetranor Prostaglandin F_{1α} methyl ester;
13,14-dihydro-15-methyl-15-methylamino-15-dehydroxy-16,16-dimethyl-16-(2-fluorophenoxy)-tetranor Prostaglandin F_{1α} methyl ester;
5,6-dihydro-15-methyl-15-methylamino-15-dehydroxy-16-methyl-16-(2-methylphenoxy)-tetranor Prostaglandin F_{1α} 1-hydroxamic acid;
13,14-dihydro-15-methyl-15-(N,N-dimethylamino)-16-ethyl-16-(2-fluorophenoxy)-tetranor Prostaglandin F_{1α} isopropyl ester; and
13,14-dihydro-16-(2,6-difluorophenoxy)-16-tetranor Prostaglandin F_{1α} glyceryl ester.

Other suitable prostaglandins of this invention include, but are not limited to: unoprostone, tafluprost; bimatoprost; cloprostenol; fluprostenol; travoprost; latanoprost; tiaprost; delprostenate; estrumate; equimate; alphaprostol; dinoprostone; dukeprost, fenprostalene, nocloprost; and their esters and amides. In another embodiment of the present invention, the prostaglandin can be selected from the group consisting of: tafluprost; bimatoprost; cloprostenol; fluprostenol; travoprost; latanoprost; tiaprost; dinoprostone; PGE1 urethral gel; unoprostone; sulprostone; dukeprost; and the alkyl esters and monoalkyl amides of these compounds.

In an embodiment of the composition of the present invention, the prostaglandin is an active ingredient formulated into a composition, such as a pharmaceutical or cosmetic composition, administered for treatment or prophylaxis of nail weakness. Standard pharmaceutical formulation techniques are used.

An embodiment of the composition of the present invention further comprises a carrier. “Carrier” means one or more compatible substances that are suitable for administration to a mammal. Carriers include, but are not limited to, solid or liquid fillers, diluents, hydrotopes, surface-active agents, and encapsulating substances. “Compatible” means that the components of the composition are capable of being commingled with the prostaglandins, and with each other, in a manner such that there is no interaction which would substantially reduce the efficacy of the composition under ordinary use situations. Carriers must be of sufficiently high purity and sufficiently low toxicity to render them suitable for administration to the mammal being treated. The carrier can be inert, or it can possess pharmaceutical benefits, cosmetic benefits, or both.

The choice of carrier for component B) depends on the route by which component A), the prostaglandins, are administered and the form of the composition. The composition can be provided in a variety of forms suitable for topical administration (e.g., local application on the skin or nails, in particular to the skin just above the cuticle, on the proximal nail fold, or directly on the nail, on the eponychium, on the Ptygerium or true cuticle, or via a liposome delivery systems, or iontophoresis). Topical administration is preferred to minimize systemic side effects.

In an embodiment of the composition of the present invention, the composition includes a carrier selected from the group consisting of water, alcohols, aloe vera gel, allantoin, glycerin, vitamin A and E oils, mineral oil, propylene, glycol, dimethyl isosorbide, PPG-2 myristyl propionate, and combinations thereof.

In an embodiment of the composition, the carrier can provide a diluent. Suitable diluents include, but not limited to, sugars such as glucose, lactose, dextrose, and sucrose; polyols such as propylene glycol; calcium carbonate; sodium carbonate; cellulose; glycerin; mannitol; and sorbitol. One of ordinary skill should understand that other diluents may be suitable without departing from the scope of the present approach.

In an embodiment of the composition of the present invention, the carrier can provide a lubricant. Suitable lubricants are typically exemplified by solid lubricants including, but not limited to, silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. One of ordinary skill should understand that other lubricants may be suitable without departing from the scope of the present approach.

In an embodiment of the composition, the carrier can provide a binder. Suitable binders include, but are not limited to, polyvinylpyrilidone; magnesium aluminum silicate; starches such as corn starch and potato starch; gelatin; tragacanth; and cellulose and its derivatives, such as sodium carboxymethylcellulose, ethyl cellulose, methylcellulose, and sodium carboxymethylcellulose. One of ordinary skill should understand that other binders may be suitable without departing from the scope of the present approach.

In an embodiment of the composition, the carrier can provide a disintegrant. Suitable disintegrants include, but are not limited to, starches, agar, alginic acid and the sodium salt thereof, effervescent mixtures, and croscarmelose. One of ordinary skill should understand that other disintegrants may be suitable without departing from the scope of the present approach.

In an embodiment of the composition of the present invention, the carrier can provide a colorant such as an FD&C dye. In any number of embodiments of the present invention, the carrier can provide a flavor such as menthol, peppermint, and fruit flavors, for example. In another embodiment, for example, the carrier can provide a sweetener such as aspartame and saccharin.

In an embodiment of the composition, the carrier can provide an antioxidant such as BHA, BHT, and vitamin E, for example.

In another embodiment of the composition, the carrier can provide a preservative such as methyl paraben and sodium benzoate, for example.

In an embodiment of the composition of the present invention, the carrier can provide a glidant such as silicon dioxide, for example.

In an embodiment of the composition, the carrier can provide a solvent, such as water, isotonic saline, ethyl oleate, alcohols such as ethanol, and phosphate buffer solutions.

In an embodiment of the composition of the present invention, the carrier can provide a suspending agent. Suitable suspending agents include, but are not limited to, cellulose and its derivatives, such as methyl cellulose and sodium carboxymethyl cellulose; AVICEL® RC-591 from FMC Corporation of Philadelphia, Pa.; tragacanth and sodium alginate.

In an embodiment of the composition, the carrier can provide a wetting agent such as lecithin, polysorbate 80, and sodium lauryl sulfate, for example.

In an embodiment of the composition, the carrier can provide a surfactant such as the TWEENS® from Atlas Powder Company of Wilmington, Del., for example.

In an embodiment of the present invention, compositions for topical administration typically comprise 0.0001 to about 1% of the prostaglandin and 90 to 99.9% of a carrier comprising: a) a diluent; b) a lubricant; c) a binder; and d) a solvent. In one embodiment of the composition, for example, the carrier comprises: a) propylene glycol; b) sesame oil; c) pyrrolidone; and d) ethanol or ethyl oleate.

The compositions of the present invention can further comprise an optional activity enhancer. The activity enhancer can be selected from the group consisting of i) nail growth stimulants (other than the prostaglandin), and ii) penetration enhancers.

In any number of embodiments of the composition of the present invention, the activity enhancer is a nail growth stimulant, other than a prostaglandin. A nail growth stimulant is exemplified by vasodilators, antiandrogens, cyclosporins, cyclosporin analogs, antimicrobials, anti-inflammatories, thyroid hormones, thyroid hormone derivatives, and thyroid hormone analogs, retinoids, triterpenes, combinations thereof, and others.

Vasodilators such as potassium channel agonists, including, but not limited to, minoxidil and minoxidil derivatives such as aminexil, cromakalin, and diazoxide, can be used as optional nail growth stimulants in the composition.

Examples of suitable antiandrogens include, but are not limited to, 5-α-reductase inhibitors such as finasteride, cyproterone acetate, azelaic acid and its derivatives, and flutamide.

Antimicrobials and antifungals include, but are not limited to, selenium sulfide, ketoconazole, triclocarbon, triclosan, zinc pyrithione, itraconazole, fluconazole, asiatic acid, hinokitiol, mipirocin, clinacycin hydrochloride, benzoyl peroxide, benzyl peroxide and minocyclin.

Examples of suitable anti-inflammatories include, but are not limited to, glucocorticoids such as hydrocortisone, mometasone furoate and prednisolone, nonsteroidal anti-inflammatories including cyclooxygenase or lipoxygenase inhibitors, and benzydamine, salicylic acid.

Triiodothyrionine is an example of a suitable thyroid hormone.

Suitable retinoids include, but are not limited to isotretinoin, acitretin, and tazarotene.

In any number of embodiments of the composition of the present invention, optional nail growth stimulants for can include, but are not limited to, benzalkonium chloride, benzethonium chloride, phenol, estradiol, chlorpheniramine maleate, chlorophyllin derivatives, cholesterol, salicylic acid, cysteine, methionine, red pepper tincture, benzyl nicotinate, D,L-menthol, peppermint oil, calcium pantothenate, panthenol, castor oil, hinokitiol, prednisolone, resorcinol, monosaccharides and esterified monosaccharides, chemical activators of protein kinase C enzymes, glycosaminoglycan chain cellular uptake inhibitors, inhibitors of glycosidase activity, glycosaminoglycanase inhibitors, esters of pyroglutamic acid, hexosaccharic acids or acylated hexosaccharic acids, aryl-substituted ethylenes, N-acylated amino acids, finasteride, flavinoids, ascomycin derivatives and analogs, histamine antagonists such as diphenhydramine hydrochloride, triterpenes such as oleanolic acid and ursolic acid, saponins, proteoglycanase or glycosaminoglycanase inhibitors, estrogen agonists, pseudoterins, cytokine and growth factor promotors, analogs or inhibitors such as interleukinl inhibitors, interleukin-6 inhibitors, interleukin-10 promotors, and tumor necrosis factor inhibitors, vitamins such as vitamin E and vitamin D analogs and parathyroid hormone antagonists, Vitamin B12 analogs and panthenol, hydroxyacids, benzophenones, and hydantoin anticonvulsants such as phenytoin, and combinations thereof.

In any number of embodiments of the composition of the present invention, the activity enhancer can be a penetration enhancer that can be added to all of the compositions for systemic administration except compositions for oral administration. The amount of the penetration enhancer, when present in the composition, is typically 1 to 5%. Examples of penetration enhancers include, but are not limited to, 2-methyl propan-2-ol; propan-2-ol; ethyl-2-hydroxypropanoate; hexan-2,5-diol; POE(2) ethyl ether; di(2-hydroxypropyl) ether; pentan-2,4-diol; acetone; POE(2) methyl ether; 2-hydroxypropionic acid; 2-hydroxyoctanoic acid; propan-1-ol, 1,4-dioxane; tetrahydrofuran; butan-1,4-diol; propylene glycol dipelargonate; polyoxypropylene 15 stearyl ether; octyl alcohol; POE ester of oleyl alcohol; oleyl alcohol; lauryl alcohol; dioctyl adipate; dicapryl adipate; di-isopropyl adipate; di-isopropyl sebacate; dibutyl sebacate; diethyl sebacate; dimethyl sebacate; dioctyl sebacate; dibutyl suberate; dioctyl azelate; dibenzyl sebacate; dibutyl phthalate; dibutyl azelate; ethyl myristate; dimethyl azelate; butyl myristate; dibutyl succinate; didecyl phthalate; decyl oleate; ethyl caproate; ethyl salicylate; isopropyl palmitate; ethyl laurate; 2-ethyl-hexyl pelargonate; isopropyl isostearate; butyl laurate; benzyl benzoate; butyl benzoate; hexyl laurate; ethyl caprate; ethyl caprylate; butyl stearate; benzyl salicylate; 2-hydroxypropanoic acid; 2-hyroxyoctanoic acid; dimethyl sulfoxide; N,N-dimethyl acetamide; N,N-dimethyl formamide; 2-pyrrolidone; 1-methyl-2-pyrrolidone; 5-methyl-2-pyrrolidone; 1,5-dimethyl-2-pyrrolidone; 1-ethyl-2-pyrrolidone; phosphine oxides; sugar esters; tetrahydrofurfural alcohol; urea; diethyl-m-toluamide; 1-dodecylazacyloheptan-2-one; and combinations thereof.

In one embodiment of the present invention, the prostaglandins are topically administered. Topical compositions that can be applied locally to the skin may be in any form including, but not limited to, solutions, oils, creams, ointments, gels, lotions, shampoos, leave-on and rinse-out nail conditioners, nail lacquers, milks, cleansers, moisturizers, sprays, skin patches, and the like. Application of the topical composition can include, but is not limited to, application on the skin just above the cuticle; application on the proximal nail fold; application directly on the nail, the eponychium, the Pytgerium, or the true cuticle; and/or application via liposome delivery systems or iontophoresis.

In an embodiment, the topical compositions of the present invention comprises: a prostaglandin as described above; and a carrier. The carrier of the topical composition preferably aids penetration of the prostaglandins into the skin to reach the environment of the nail bed. The carrier can further comprise one or more optional components. In an embodiment of the topical composition, one or more of the optional activity enhancers described above are also included in the composition.

The exact amounts of each component in the topical composition depend on various factors. The amount of prostaglandin used in an embodiment of the composition depends on the activity of the prostaglandin selected. For example, if the binding affinity of the prostaglandin at the nail-growth activating receptor is 1 nM, the amount of prostaglandin used can be 0.0001 to 0.01%. If the binding affinity of the prostaglandin is 10 nM, the amount of prostaglandin used can be 0.001 to 0.1%. If the binding affinity of the prostaglandin is 100 nM, the amount of prostaglandin used can be 0.01 to 1.0%. If the binding affinity of the prostaglandin is 1000 nM, the amount of prostaglandin used can be 0.1 to 10%, preferably 0.5 to 5%. The amount and dosage of the prostaglandin are critical. If the amount of the prostaglandin used is outside the ranges specified above (i.e., either higher or lower), efficacy of the treatment can be reduced, although the composition can still be effective.

In any number of embodiments, the topical composition can further comprise 1 to 20% of an optional activity enhancer, and a sufficient amount of a carrier, such that the amounts of the prostaglandin, the optional activity enhancer and the carrier, combined equal 100%. The amount of the carrier employed in conjunction with the prostaglandin is sufficient to provide a practical quantity of material for administration per unit dose of the compound. Techniques and compositions for making dosage forms useful in the methods of this invention are well known in the field.

The carrier can comprise a single component or a combination of two or more components. Typical carriers used in the topical compositions include, but are not limited to, water, alcohols, aloe vera gel, allantoin, glycerin, vitamin A and E oils, mineral oil, propylene glycol, PPG-2 myristyl propionate, dimethyl isosorbide, combinations thereof, and the like. Preferred carriers include propylene glycol, dimethyl isosorbide, and water.

An embodiment of the carrier of the topical composition may further comprise one or more ingredients selected from the group consisting of: emollients; propellants; solvents; humectants; thickeners; powders; and fragrances.

In an embodiment of the composition wherein the carrier includes an emollient, the amount of the emollient used in the topical composition can typically be 5 to 95%. Suitable emollients include, but are not limited to, stearyl alcohol; glyceryl monoricinoleate; glyceryl monostearate; propane-1,2-diol; butane-1,3-diol; mink oil; cetyl alcohol; iso-propyl isostearate; stearic acid; iso-butyl palmitate; isocetyl stearate; oleyl alcohol; isopropyl laurate; hexyl laurate; decyl oleate; octadecan-2-ol; isocetyl alcohol; cetyl palmitate; polydimethylsiloxane; di-n-butyl sebacate; iso-propyl myristate; iso-propyl palmitate; iso-propyl stearate; butyl stearate; polyethylene glycol; triethylene glycol; lanolin; sesame oil; coconut oil; arachis oil; castor oil; acetylated lanolin alcohols; petroleum; mineral oil; butyl myristate, isostearic acid, palmitic acid, isopropyl linoleate, lauryl lactate, myristyl lactate; decyl oleate; myristyl myristate; and combinations thereof. Preferred emollients include stearyl alcohol and polydimethylsiloxane.

In an embodiment of the present invention wherein the carrier includes a propellant, the amount of the propellant used in the topical composition can typically be 5 to 95%. Suitable propellants include, but are not limited to, propane, butane, isobutane, dimethyl ether, carbon dioxide, nitrous oxide, and combinations thereof.

In an embodiment of the present invention wherein the carrier includes a solvent, the amount of the solvent used in the topical composition can typically be 5 to 95%. Suitable solvents include, but are not limited to, water, ethyl alcohol, methylene chloride, isopropanol, castor oil, ethylene glycol monoethyl ether, diethylene glycol monobutyl ether, diethylene glycol monoethyl ether, dimethyl sulfoxide, dimethyl formamide, tetrahydrofuran, and combinations thereof. Preferred solvents include ethyl alcohol.

In an embodiment of the present invention wherein the carrier includes a humectant, the amount of the solvent used in the topical composition can typically be 5 to 95%. Suitable humectants include, but are not limited to, glycerin, sorbitol, sodium 2-pyrrolidone-5-carboxylate, soluble collagen, dibutyl phthalate, gelatin, and combinations thereof. Preferred humectants include glycerin.

In an embodiment of the present invention wherein the carrier includes a thickener, the amount of the thickener used in the topical composition can typically be 0 to 95%.

In an embodiment of the present invention wherein the carrier includes a powder, the amount of the powder used in the topical composition can typically be 0 to 95%. Suitable powders include, but are not limited to, chalk, talc, Fuller's earth, kaolin, starch, gums, colloidal silicon dioxide, sodium polyacrylate, tetra alkyl ammonium smectites, trialkyl aryl ammonium smectites, chemically modified magnesium aluminum silicate, organically modified montmorillonite clay, hydrated aluminum silicate, fumed silica, carboxyvinyl polymer, sodium carboxymethyl cellulose, ethylene glycol monostearate, and combinations thereof.

In an embodiment of the present invention wherein the carrier includes a fragrance, the amount of the fragrance used in the topical composition can typically be 0.001 to 0.5%. In another embodiment, the amount of fragrance used in the topical composition can be 0.001 to 0.1%.

In any number of embodiments, the topical composition of the present invention further comprises an optional activity enhancer as described above. Any of the i) activity enhancers and ii) penetration enhancers may be added to the topical compositions. In one embodiment, the topical composition comprises 0.01 to 15% of an activity enhancer, an optional nail growth stimulant. In another embodiment, the composition comprises 0.1 to 10% of an activity enhancer, an optional nail growth stimulant. In another embodiment, the topical composition comprises 0.5 to 5% of an activity enhancer, an optional nail growth stimulant. In one embodiment, the topical composition comprises 1 to 5% of component ii), a penetration enhancer.

In an alternative embodiment of the composition, topical pharmaceutical compositions for ocular administration are prepared by conventional methods. Topical pharmaceutical compositions for ocular administration typically comprise A) a prostaglandin; and B) a carrier, wherein the carrier includes a base such as purified water, and one or more ingredients selected from the group consisting of: (i) sugars such as dextrans, particularly dextran 70; (ii) cellulose or a derivative thereof; (iii) a salt; (iv) disodium EDTA (Edetate disodium); and (v) a pH adjusting additive.

Examples of cellulose derivatives suitable for use in the topical pharmaceutical composition for ocular administration include, but are not limited to, sodium carboxymethyl cellulose, ethyl cellulose, methyl cellulose, and hydroxypropylmethylcellulose. Hydroxypropyl-methylcellulose is preferred.

Examples of salts suitable for use in the topical pharmaceutical composition for ocular administration include, but are not limited to, sodium chloride, potassium chloride, and combinations thereof.

Examples of pH adjusting additives include, but are not limited to, HCl or NaOH in amounts sufficient to adjust the pH of the topical pharmaceutical composition for ocular administration to 7.2-7.5.

In any number of embodiments of the composition, the prostaglandins can be administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines.

In any number of embodiments of the present invention, the prostaglandins can be administered by iontophoresis.

In any number of embodiments, the prostaglandins can be included in kits comprising a prostaglandin, a topical composition described above, or both; and information, instructions, or both that use of the kit can provide treatment for nail weakness or to enhance nail thickness, integrity, strength, growth rate and/or appearance in mammals (particularly humans). The information and instructions can be in the form of words, pictures, or both, and the like. In addition, or in the alternative, the kit can comprise a prostaglandin, a composition, or both; and information, instructions, or both, regarding methods of application of the prostaglandin or composition, preferably with the benefit of treating nail weakness in mammals.

In all of the foregoing compositions, and for all routes of administration, the prostaglandins can be used alone or in combinations of two or more prostaglandins. The compositions can further comprise additional drugs or excipients as appropriate for the indication, such as an antifungal agent if there is a nail fungal infection present.

### Methods

Disclosed is a method for treating nail weakness or enhancing nail thickness, integrity, strength, growth rate and/or appearance in mammals. An embodiment of the method comprises administering to a mammal (preferably a human) suffering from nail weakness or less than desired nail thickness, integrity, growth rate or appearance, a prostaglandin as described above. For example, a human diagnosed with weak, brittle or peeling nails can be treated by the methods of this invention. In one embodiment, a systemic or topical composition comprising: A) a prostaglandin; and B) a carrier can be administered to the mammal. In another embodiment, the composition is a topical composition comprising: A) a prostaglandin; B) a carrier; and C) an optional activity enhancer.

The dosage of the prostaglandin administered depends on the method of administration, for example. In an embodiment for systemic administration, (e.g., oral, rectal, nasal, sublingual, buccal, or parenteral), typically, 0.5 mg to 300 mg, preferably 0.5 mg to 100 mg, more preferably 0.1 mg to 10 mg, of a prostaglandin described above can be administered per day. These dosage ranges are merely exemplary, and daily administration can be adjusted. In many cases, systemic administration is not recommended, depending on various factors. The specific dosage of the prostaglandin to be administered, as well as the duration of treatment, and whether the treatment is topical or systemic are interdependent. The dosage and treatment regimen can also depend upon such factors as the specific prostaglandin used, the treatment indication, the efficacy of the compound, the personal attributes of the subject (such as, for example, weight, age, sex, and medical condition of the subject), compliance with the treatment regimen, and the presence and severity of any side effects of the treatment, for example.

In an embodiment for topical administration (e.g., local application on the skin or nails, in particular to the skin just above the cuticle, on the proximal nail fold, or directly on the nail, on the eponychium, on the ptygerium or true cuticle, or via a liposomal delivery systems, or by iontophoresis), the topical composition can typically be administered once per day for up to six months. Generally, 12-24 weeks is sufficient to see enhanced effects, and approximately 8 months to achieve complete turnover of the fingernail, longer for toenails, but treatment may be extended indefinitely to maintain the health and growth of the nails.

### EXAMPLES

These non-limiting examples are intended to illustrate the invention to those skilled in the art and should not be interpreted as limiting the scope of the invention set forth in the claims.

### Reference Example 1

### Analytical Methods

Prostaglandins are tested for their potential to grow nails using in vitro assays as an in vivo assay for nails has yet to be accepted in the literature.

Without intending to be limited by theory, there are two principal prostaglandin receptor families that control nail growth: the Prostanoid F receptor family and the Prostanoid E1 receptor family. Potential at these receptors is measured in two types of assays-binding assays and functional assays. The binding assay is described herein and the functional assay follows as Reference Example 2.

Competition Binding Assays:

The compounds were evaluated for their ability to displace a radiolabelled prostaglandin in membrane preparations isolated from CHO-KI cells stably transfected with the hFP prostaglandin receptor. Competition assays were performed to determine the IC₅₀ of the compounds. The [³H]PG F_{2α} (5 nM), isolated membrane and compounds incubated together for one hour, before the plates were harvested and the DPM's were counted.

In detail, COS 7 (SV40 transformed; African Green Monkey kidney cell line)(American Type Culture Collection, VWR Scientific) were transfected with hFP receptor plasmid DNA. The DNA vector used is pCR 3.1, and the plasmid is in E. coli INVF′ competent cells (Invitrogen). The hFP receptor plasmid DNA was isolated using a Qiagen Giga DNA preparation kit. The COS 7 cells were grown in T150 flasks until 80-90% confluent. At this point, the cells were transfected with the hFP receptor plasmid DNA using the following protocol for each T150 flask: Two ml of plain DMEM was mixed with 11 ug DNA and 55 ul of lipofectamine plus reagent. This was allowed to sit for 15 minutes at room temperature. In another container, 2 mL of plain DMEM was mixed with 82 ul of lipofectamine. The lipofectamine mixture was added to the DNA mixture after the 15 minute incubation period. This new mixture was allowed to incubate 15 minutes more. Then 14 mL of plain DMEM was added to the reaction mixture. The COS 7 growth media was emptied out of the T150 flask and was replaced by the DNA/lipofectamine mixture. The flask was incubated for 3 hours at 37° C. After 3 hours, the DNA/lipofectamine mixture was emptied out and replaced with 30 ml of 10% Calf Serum in DMEM. The flask sat for 2 days at 37° C.

After the 2 days of incubation, the cell membranes were isolated using the following procedure: The cells were washed with 25 ml of cold Hank's Buffered Saline Solution (HBSS). Then 15 ml of versene was added to the empty flask and was allowed to sit 15 minutes. The flask was tapped to dislodge the cells from the flask and 25 mL of HBSS was added to the flask to rinse the flask. The cells were poured into a 50 mL conical vial and centrifuged for 10 min, 4° C. at 2000 RPM. The supernatant was poured off and cold Phosphate Buffered Saline (PBS)/EDTA, pH 7.4, was added, 3 ml per flask of cells collected. The cells were placed in a nitrogen bomb (model 4639) for 15 minutes, 800 psi. The cells were collected and spun at 3000 RPM, 20 minutes, 4° C. The supernatant collected was centrifuged again in a Beckman XL-80 Ultracentrifuge at 33,000 RPM, 1 hour, 4° C. The pelleted hFP membrane receptor was resuspended in TME (tris-HCL, MgCl₂, EDTA) buffer, pH 6.0, about 2 mL for every 10 flasks of cells collected. A polytron was used to homogenize the membrane. A BCA protein assay (Pierce BCA kit) was used to determine the amount of receptor collected so that the volume could be adjusted to 1 mg/mL to be frozen at −80° C.

An initial competition binding assay was done in a 2 mL deep well plate to determine how much receptor was needed to run the compound screening competition binding assays. The general procedure for this is as follows: Various amounts of hFP receptor (10 ug to 100 ug) were combined with 50 ul (5 nM) of [³H]PG F_{2α} (Amersham) and 50 ul of a TME/ethanol (B/E) solution. TME buffer is used to bring the volume in each well up to 200 uL. The B/E solution is used as the vehicle control. These are the Total Binding, which are done in triplicate. Non-specific Binding (NSB) is also done in triplicate and the same amounts of receptor, TME buffer and [³H]PG F_{2α} are added to the wells. Instead of B/E solution, 50 uL of a 1E-5 M of cold PG F_{2α} ligand is added. The reaction mixtures are allowed to incubate for 1 hour at 23 OC. The reaction is filtered through GF/C filters. Forty uL of scintillation cocktail was added to each well of the filter plate then counted after 3 hours of incubation using an Hewlett Packard top count. A linear regression curve was generated from the DPM results and the amount of receptor needed for future assays will be determined from this. The difference of the Total and the NSB will be the Specific Binding. The amount of receptor used must have the minimum amount of NSB DPM counts and an amount of about 5000 DPM counts of specific binding.

Compound screening competition binding assays were done for compound plates that have 96 wells of compounds with concentrations of 10 mM. A daughter plate was made (1:50 dilution) to get a final concentration of 200 uM. Fifty uL of the compounds from the daughter plate were added to triplicate 2 ml deep well reaction plates. Fifty uL of 5 nM [³H]PG F_{2α} (Amersham) and the appropriate amount of hFP receptor (previously determined) was added to each well of the triplicate reaction plates. TME buffer was added to all of the plates to bring each well volume up to 200 uL. The final concentrations of the compounds in each well were 50 uM. Triplicate positive controls were done at concentrations of cloprostenol, final concentrations ranging from 1E-5 to 1E-10 M. The plates were incubated for 1 hour at 23° C. then filtered onto GF/C filters. Forty uL of scintillation cocktail was added to each well, incubated for 3 hours then counted using a Hewlett Packard topcount. The cloprostenol control DPMs were plotted and an EC₅₀ was calculated to ensure that the control bound with expected results. Triplicate DPMs for each individual compound were evaluated at this point.

### Reference Example 2

### Analytical Methods

Prostaglandins are tested for their potential to grow nails using in vitro assays as a definitive in vivo assay for nails has yet to be accepted in the literature.

Without intending to be limited by theory, there are two principal prostaglandin receptor families that control nail growth: the Prostanoid F receptor family and the Prostanoid E1 receptor family. Potential at these receptors in measured in two types of assays-binding assays and functional assays. The functional assay is described herein and the binding assay is above as Reference Example 1.

There are many functional assays available, for example, growth in Swiss 3T3 cells. Preferred is the R-SAT system. R-SAT is a cell-based assay system where genes are transferred to cultured cells. The functional activity of the gene products, or potential drug targets, are then evaluated through signal transduction pathways that lead to cellular growth. The growth signals are reported using marker gene technologies. Thus, effects of drugs on potential drug targets can be efficiently detected as changes in color or fluorescence. R-SAT enables the efficient screening of large compound libraries for identification of new chemistries at given targets, as well as detailed pharmacological testing of compounds at a wide range of targets, as described in MESSIER, T., DORMAN, C. M.; BRAUNEROSBORNE, H.; EUBANKS, D. & BRANN, M. R. (1995) Pharmacol. and Toxicol. 76 308-311, incorporated herein by reference.

### Example 1

A prostaglandin having the structure:

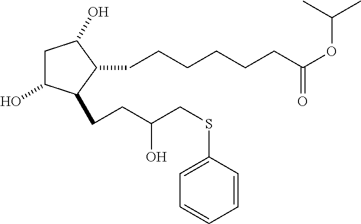 [SMILES: CC(C)OC(=O)CCCCCC[C@@H]1[C@@H](CCC(O)CSc2ccccc2)[C@H](O)C[C@@H]1O]

was tested according to the method of Reference Example 1.

### Example 2

A prostaglandin having the structure:

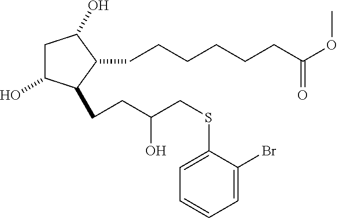 [SMILES: COC(=O)CCCCCC[C@@H]1[C@@H](CCC(O)CSc2ccccc2Br)[C@H](O)C[C@@H]1O]

was tested according to the method of Reference Example 2.

TABLE 3
Average Grades

| Example | 23 Days | 25 Days | 26 Days |
|---|---|---|---|
| 1 | 0.4 | 0.1 | 0.7 |
| 2 | 0.1 | not measured | not measured |

### Example 3

Compositions for topical administration are made, comprising:

| Component | 3-1 | 3-2 | 3-3 | 3-4 |
|---|---|---|---|---|
| prostaglandin (wt %) | 0.01 | 0.1 | 1.0 | 10.0 |
| ~IC₅₀ the prostaglandin (nM) | 1 | 10 | 100 | 1000 |
| Ethanol (wt %) | 59.99 | 59.9 | 59.4 | 54.0 |
| Propylene Glycol (wt %) | 20.00 | 20.0 | 19.8 | 18.0 |
| Dimethyl Isosorbide (wt %) | 20.00 | 20.0 | 19.8 | 18.0 |

A human female subject suffering from nail weakness is treated by a method of this invention. Specifically, for 6 months, one of the above compositions is daily administered topically to the cuticles of each of the fingernails and toenails of the subject.

### Example 4

A composition for topical administration is made according to the method of Dowton et al., “Influence of Liposomal Composition on Topical Delivery of Encapsulated Cyclosporin A: I. An in vitro Study Using Mouse Skin”, S.T.P. Pharma Sciences, Vol. 3, pp. 404-407 (1993), using a prostaglandin in lieu of cyclosporin A and using the NOVASOME® 1 (available from Micro-Pak, Inc. of Wilmington, Del.) for the non-ionic liposomal formulation.

A human male subject suffering thin, splitting and peeling nails is treated each day with the above composition. Specifically, for 6 months, the above composition is administered topically to the subject at the base of each fingernail.

### Example 5

Compositions for topical administration are made, comprising Bimatoprost Ophthalmic Solution, with the active ingredient of (Z)-7-[(1R,2R,3R,5S)-3,5-Dihydroxy-2-[(1E,3S)-3-hydroxy-5-phenyl-1-pentenyl]cyclopentylj-5-N-ethylheptenamide (bimatoprost).

A human female subject suffering from nail weakness is treated by a method of this invention. Specifically, for 6 months, the above composition is daily administered topically to the cuticles of each of the fingernails and toenails of the subject.

### Example 6

Compositions for topical administration are made, comprising Travoprost Ophthalmic Solution, with the active ingredient of [1R-[1α(Z),2β(1E,3R*),3α,5α]]-7-[3,5-Dihydroxy-2-[3-hydroxy-4-[3-(trifluoromethyl)phenoxy]-1-butenyl]cyclopentyl]-5-heptenoic acid, 1-methylethylester (travoprost).

A human male subject suffering from slow nail growth is treated by a method of this invention. Specifically, for 6 months, the above composition is daily administered topically to the cuticles of each of the fingernails and toenails of the subject.

### Example 7

Compositions for topical administration are made, comprising Bimatoprost Ophthalmic Solution, with the active ingredient of isopropyl-(Z)-7[(1R,2R,3R,5S)3,5-dihydroxy-2-[(3R)-3-hydroxy-5-phenylpentyl]cyclopentyl]-5-heptenoate. (latanoprost).

A human female subject suffering from nail weakness is treated by a method of this invention. Specifically, for 6 months, one drop of the above composition is daily administered topically to the cuticles the affected fingernails and toenails of the subject.

### Example 8

Compositions for topical administration are made, comprising Tafluprost Ophthalmic Solution, with the active ingredient of (5Z)-7-[(1R,2R,3R,5S)-2-[(1E)-3,3-Difluoro-4-phenoxy-1-buten-1-yl]-3,5-dihydroxycyclopentyl]-5-heptenoic acid 1-methylethyl ester (tafluprost).

A human male subject suffering from nail brittleness and peeling is treated by a method of this invention. Specifically, for 6 months, one drop of the above composition is daily administered topically to either the skin just above the cuticle, on the proximal nail fold, directly on the nail, on the eponychium, or on the Ptygerium or true cuticle, of the affected fingernails and toenails of the subject.

### Example 9

Compositions for topical administration are made, comprising Cloprostenol for Injection Solution, with the active ingredient of Sodium [1alpha(Z),2beta(1E,3R*), 3alpha,5alpha]-(+/−)-7-[2-[4-(3-chlorophenoxy)-3-hydroxybut-1-enyl]-3,5-dihydroxycyclopentyl]hept-5-enoate (cloprostenol sodium).

A human male subject suffering from nail fragility is treated by a method of this invention. Specifically, for 6 months, one drop of the above composition is daily administered topically to the cuticles the affected fingernails and toenails of the subject.

### Example 10

Compositions for topical administration are made, comprising Cloprostenol for Injection Solution, with the active ingredient of Sodium (5Z)-rel-7-[(1R,2R,3R,5S)-3,5-Dihydroxy-2-[(1E,3R)-3-hydroxy-4-[3-(trifluoromethyl)phenoxy]-1-butenyl]cyclopentyl]-5-heptenoate (fluprostenol sodium).

A human male subject suffering from nail weakness and slow growth is treated by a method of this invention. Specifically, for 12 months, one drop of the above composition is daily administered topically to the cuticles the affected fingernails and toenails of the subject.

### Example 11

Compositions for topical administration are made, comprising unoprostone isopropyl 0.15% ophthalmic solution, with the active ingredient of 13,14-Dihydro-15-keto-20-ethyl-PGF1α; also known as (5Z)-7-((1R,2R,3R,5S)-3,5-Dihydroxy-2-(3-oxodecyl)cyclopentyl)-5-heptenoic acid 1-methylethyl ester (isopropyl unoprostone).

A human male subject suffering from nail splitting and slow growth is treated by a method of this invention. Specifically, for 12 months, one drop of the above composition is daily administered topically to the cuticles the affected fingernails and toenails of the subject.

While the invention has now been described in terms of a preferred embodiment of compositional ranges and a preferred treatment method, the skilled artisan will appreciate the various substitutions, modifications, changes, and omissions may be made without departing from the spirit thereof.

The following claims represent additional disclosure and in no way are intended to represent a legal definition of the scope of the present invention.

We claim:

1. A topical composition for treating nails, the topical composition comprising:
A) an active ingredient selected from the group consisting of a prostaglandin analog having the structure: 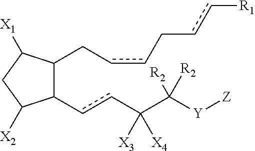
and
B) a carrier selected from the group consisting of water, alcohols, aloe vera gel, allantoin, glycerin, vitamin A and E oils, mineral oil, propylene glycol, dimethyl isosorbide, PPG-2 myristyl propionate, and combinations thereof.

2. The topical composition of claim 1, wherein R¹ is selected from the group consisting of CO₂H, C(O)NHR⁵, CO₂R⁵, CH₂OH, S(O)₂R⁵, P(O)MeOR⁵, C(O)NHR⁵, C(O)NHS(O)₂R⁵, and tetrazole.

3. The topical composition of claim 1, wherein R¹ is selected from the group consisting of CO₂H, CO₂CH₃, CO₂C₂H₅, CO₂C₃H₇, CO₂C₄H₉, CO₂C₃H₇O₂, and C(O)NHR⁵.

4. The topical composition of claim 2, wherein R⁵ is selected from the group consisting of monovalent hydrocarbon groups, substituted monovalent hydrocarbon groups, aromatic groups, substituted aromatic groups, carbocyclic groups, substituted carbocyclic groups, heterogeneous groups, substituted heterogeneous groups, heterocyclic groups, substituted heterocyclic groups, heteroaromatic groups, and substituted heteroaromatic groups.

5. The topical composition of claim 2, wherein R⁵ is selected from the group consisting of CH₃, C₂H₅, and C₃H₇.

6. The topical composition of claim 3, wherein R⁵ is selected from the group consisting of monovalent hydrocarbon groups, substituted monovalent hydrocarbon groups, aromatic groups, substituted aromatic groups, carbocyclic groups, substituted carbocyclic groups, heterogeneous groups, substituted heterogeneous groups, heterocyclic groups, substituted heterocyclic groups, heteroaromatic groups, and substituted heteroaromatic groups.

7. The topical composition of claim 3, wherein R⁵ is selected from the group consisting of CH₃, C₂H₅, and C₃H₇.

8. The topical composition of claim 1, wherein R² is selected from the group consisting of a hydrogen atom and a methyl group.

9. The topical composition of claim 1, wherein each R² is independently a hydrogen atom or a lower monovalent hydrocarbon group.

10. The topical composition of claim 1, wherein X₁, X₂, X₃ and X₄ are each independently selected from the group consisting of H, halogen, CH₃, C₂H₅, NR²R², OR¹⁰, SR¹⁰, and OH; and when X₃ is OH, X₄ is selected from the group consisting of H, halogen, CH₃, C₂H₅, NR²R², OR¹⁰, and SR¹⁰.

11. The topical composition of claim 10, wherein R¹⁰ is selected from the group consisting of a monovalent hydrocarbon group, a substituted monovalent hydrocarbon group, a heterogeneous group, a substituted heterogeneous group, a carbocyclic group, a substituted carbocyclic group, an aromatic group, a substituted aromatic group, a heteroaromatic group, and a substituted heteroaromatic group; and R¹⁰ has 1 to 8 member atoms.

12. The topical composition of claim 1, wherein Y is selected from the group consisting of an oxygen atom, a divalent hydrocarbon group, a sulfur-containing moiety, and a nitrogen-containing group.

13. The topical composition of claim 12, wherein the divalent hydrocarbon group has the formula (CH₂)ₙ, wherein n is an integer with a value of 0 to 5.

14. The topical composition of claim 12, wherein the sulfur-containing moiety for Y is selected from the group consisting of a sulfur atom, S(O), and S(O)₂.

15. The topical composition of claim 12, wherein the nitrogen-containing group for Y has the formula NR¹¹.

16. The topical composition of claim 15, wherein R¹¹ is selected from the group consisting of a hydrogen atom, an acyl group, a monovalent hydrocarbon group, a substituted monovalent hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group.

17. The topical composition of claim 1, wherein Z is selected from the group consisting of a hydrogen atom, an alkyl group, carbocyclic group, a substituted carbocyclic group, a heterocyclic group, a substituted heterocyclic group, an aromatic group, a substituted aromatic group, a heteroaromatic group, and a substituted heteroaromatic group.

18. The topical composition of claim 1, wherein Z is selected from the group consisting of a monocyclic carbocyclic group, a substituted monocyclic carbocyclic group, a monocyclic heterocyclic group, a substituted monocyclic heterocyclic group, a monocyclic aromatic group, a substituted monocyclic aromatic group, a monocyclic heteroaromatic group, and a substituted monocyclic heteroaromatic group.

19. The topical composition of claim 1, further comprising an optional activity enhancer selected from the group consisting of i) nail growth stimulants and ii) penetration enhancers.

20. A method of treating nail weakness, wherein the method comprises administering to a mammal a composition comprising:
A) an active ingredient selected from the group consisting of a prostaglandin analog having the structure: 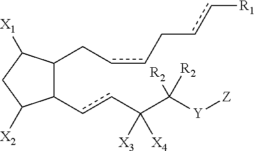
and
B) a carrier selected from the group consisting of water, alcohols, aloe vera gel, allantoin, glycerin, vitamin A and E oils, mineral oil, propylene glycol, dimethyl isosorbide, PPG-2 myristyl propionate, and combinations thereof.

21. The method of claim 20, wherein the composition is a topical composition locally administered to the area of the nail at least once per day.

22. The method of claim 20, wherein the composition is administered at least once per day for 6 to 12 months.

23. The method of claim 20, wherein R¹ is selected from the group consisting of CO₂H, C(O)NHR⁵, CO₂R⁵, CH₂OH, S(O)₂R⁵, P(O)MeOR⁵, C(O)NHR⁵, C(O)NHS(O)₂R⁵, and tetrazole.

24. The method of claim 20, wherein R¹ is selected from the group consisting of CO₂H, CO₂CH₃, CO₂C₂H₅, CO₂C₃H₇, CO₂C₄H₉, CO₂C₃H₇O₂, and C(O)NHR⁵.

25. The method of claim 23, wherein R⁵ is selected from the group consisting of monovalent hydrocarbon groups, substituted monovalent hydrocarbon groups, aromatic groups, substituted aromatic groups, carbocyclic groups, substituted carbocyclic groups, heterogeneous groups, substituted heterogeneous groups, heterocyclic groups, substituted heterocyclic groups, heteroaromatic groups, and substituted heteroaromatic groups.

26. The method of claim 23, wherein R⁵ is selected from the group consisting of CH₃, C₂H₅, and C₃H₇.

27. The method of claim 24, wherein R⁵ is selected from the group consisting of monovalent hydrocarbon groups, substituted monovalent hydrocarbon groups, aromatic groups, substituted aromatic groups, carbocyclic groups, substituted carbocyclic groups, heterogeneous groups, substituted heterogeneous groups, heterocyclic groups, substituted heterocyclic groups, heteroaromatic groups, and substituted heteroaromatic groups.

28. The method of claim 24, wherein R⁵ is selected from the group consisting of CH₃, C₂H₅, and C₃H₇.

29. The method of claim 20, wherein R² is selected from the group consisting of a hydrogen atom and a methyl group.

30. The method of claim 20, wherein each R² is independently a hydrogen atom or a lower monovalent hydrocarbon group.

31. The method of claim 20, wherein X₁, X₂, X₃ and X₄ are each independently selected from the group consisting of H, halogen, CH₃, C₂H₅, NR²R², OR¹⁰, SR¹⁰, and OH; and when X₃ is OH, X₄ is selected from the group consisting of H, halogen, CH₃, C₂H₅, NR²R², OR¹⁰, and SR¹⁰.

32. The method of claim 31, wherein R¹⁰ is selected from the group consisting of a monovalent hydrocarbon group, a substituted monovalent hydrocarbon group, a heterogeneous group, a substituted heterogeneous group, a carbocyclic group, a substituted carbocyclic group, an aromatic group, a substituted aromatic group, a heteroaromatic group, and a substituted heteroaromatic group; and R¹⁰ has 1 to 8 member atoms.

33. The method of claim 20, wherein Y is selected from the group consisting of an oxygen atom, a divalent hydrocarbon group, a sulfur-containing moiety, and a nitrogen-containing group.

34. The method of claim 33, wherein the divalent hydrocarbon group has the formula (CH₂)ₙ, wherein n is an integer with a value of 0 to 5.

35. The method of claim 33, wherein the sulfur-containing moiety for Y is selected from the group consisting of a sulfur atom, S(O), and S(O)₂.

36. The method of claim 33, wherein the nitrogen-containing group for Y has the formula NR¹¹.

37. The method of claim 36, wherein R¹¹ is selected from the group consisting of a hydrogen atom, an acyl group, a monovalent hydrocarbon group, a substituted monovalent hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group.

38. The method of claim 20, wherein Z is selected from the group consisting of a hydrogen atom, an alkyl group, carbocyclic group, a substituted carbocyclic group, a heterocyclic group, a substituted heterocyclic group, an aromatic group, a substituted aromatic group, a heteroaromatic group, and a substituted heteroaromatic group.

39. The method of claim 20, wherein Z is selected from the group consisting of a monocyclic carbocyclic group, a substituted monocyclic carbocyclic group, a monocyclic heterocyclic group, a substituted monocyclic heterocyclic group, a monocyclic aromatic group, a substituted monocyclic aromatic group, a monocyclic heteroaromatic group, and a substituted monocyclic heteroaromatic group.

40. The method of claim 20, further comprising an optional activity enhancer selected from the group consisting of i) nail growth stimulants and ii) penetration enhancers.
